(19) United States
(12) Patent Application Publication
(10) Pub. No.: US 2022/0047471 A1
(43) Pub. Date: Feb. 17, 2022

(54) WATER-IN-OIL EMULSION COSMETIC

(71) Applicant: Shiseido Company, Ltd., Chuo-ku, Tokyo (JP)

(72) Inventors: Yukari SAITO, Tokyo (JP); Tomoko IKEDA, Tokyo (JP)

(73) Assignee: Shiseido Company, Ltd., Chuo-ku, Tokyo (JP)

(21) Appl. No.: 17/414,538

(22) PCT Filed: Nov. 18, 2019
(86) PCT No.: PCT/JP2019/045030

§ 371 Date: Jun. 16, 2021

(30) Foreign Application Priority Data
Dec. 19, 2018 (JP) 2018-236997

(51) Int. Cl. A61K 8/06; A61K 8/36; A61K 8/73; A61K 8/891; A61K 8/86; A61Q 19/00

(52) U.S. Cl. CPC A61K 8/064; A61K 8/361; A61K 8/731; A61K 8/891; A61K 2800/413; A61Q 19/00; A61K 2800/48; A61K 2800/594; A61K 2800/10; A61K 8/86

(57) ABSTRACT

A water-in-oil emulsifying cosmetic that has a smooth texture while suppressing stickiness, and that further has an excellent fitting sensation when applied. The water-in-oil emulsion cosmetic of the present invention contains (A) a water-soluble thickener in an internal phase; and contains at least one of (B) an emulsifying crosslinked elastomer and (C) a metal soap in an external phase.

### TECHNICAL FIELD

The present invention relates to a water-in-oil emulsion cosmetic. More specifically, the present invention relates to a water-in-oil emulsion cosmetic that is not sticky, that has an excellent fitting sensation when applied, and that also has a smooth texture.

### BACKGROUND ART

Water-in-oil emulsion-type cosmetics have excellent water resistance in comparison to those of the oil-in-water emulsion type, and also have the characteristic that emollient oils, oil-soluble medicinal agents, ultraviolet absorption agents and the like can be efficiently blended therein, and thus can be formulated into cosmetics with high skin-care effects. However, water-in-oil emulsion cosmetics have poor stability. Thus, in order to improve the dispersion stability, it is common to add an oil-soluble thickener such as an organically modified clay mineral or a dextrin fatty acid ester, thereby thickening the oils constituting the external phase (oil phase) and encapsulating the internal phase (water phase) therein. However, there is a problem in that adding an oil-soluble thickener reduces the sensation of freshness, and causes stickiness and oiliness.

Meanwhile, improving the stability of emulsion particles by blending a water-soluble thickener into the internal phase (water phase) of a water-in-oil emulsion cosmetic has also been proposed.

For example, Patent Document 1 discloses a water-in-oil emulsion cosmetic containing prescribed amounts of a specific diester, a non-ionic surfactant, an oil-soluble and/or water-soluble thickener, a powder, and water. The stability is increased by using a single or a combination of multiple oil-soluble and/or water-soluble thickeners.

Patent Document 2 discloses a water-in-oil emulsion cosmetic containing a silicone-based surfactant, an oil, an oil gelling agent, a water-soluble polymer thickener, and water. An appropriate level of viscoelasticity is imparted to the water phase by blending in a water-soluble polymer thickener, thereby obtaining a light and non-sticky feel.

Patent Document 3 discloses a water-in-oil emulsion cosmetic characterized by containing fine particles of at least one type selected from the group consisting of cellulose fine particles and cellulose composite fine particles having an average particle size of 10 to 500 nm, obtained by physically treating the cellulose so as to be finer. The stability over time and the spreadability are improved by dispersing the fine particles in the water phase.

However, when a water-soluble thickener is blended into the water-in-oil emulsion cosmetic, the lack of stickiness is improved but is not adequate, and it is difficult to obtain satisfactory results in terms of the smoothness, the fitting sensation and the like in comparison to the case in which an oil-soluble thickener is blended. Thus, further improvements are sought.

### RELATED ART

### Patent Documents

### Patent Document 1: JP 2010-150184 A

### Patent Document 2: JP 2008-127306 A

### Patent Document 3: JP 2006-342140 A

### SUMMARY OF THE INVENTION

### Problem to be Solved by the Invention

The present invention was made in consideration of the aforementioned circumstances, and an objective of the invention is to provide a water-in-oil emulsion cosmetic that has a smooth texture while suppressing stickiness, and that further has an excellent fitting sensation when applied.

### Means for Solving the Problem

As a result of performing diligent research towards solving the above-mentioned problem, the present inventors discovered that the aforementioned problem can be solved by blending a water-soluble thickener into the internal phase (water phase) of a water-in-oil emulsion cosmetic, and also blending at least one of an emulsifying crosslinked elastomer and a metal soap into the external phase (oil phase), thereby completing the present invention.

As mentioned above, when just a water-soluble thickener is blended into a water-in-oil emulsion cosmetic, the lack of stickiness is improved but is not adequate, and it was difficult to obtain satisfactory results in terms of the smoothness, the fitting sensation and the like. The present inventors discovered that, by further blending at least one of an emulsifying crosslinked elastomer and a metal soap in addition to a water-soluble thickener, the smoothness can be improved while suppressing stickiness, and a water-in-oil emulsion cosmetic that also has an excellent fitting sensation when applied can be obtained.

In other words, the present invention provides a water-in-oil emulsion cosmetic that contains (A) a water-soluble thickener in an internal phase; and contains at least one of (B) an emulsifying crosslinked elastomer and (C) a metal soap in an external phase.

### Effects of the Invention

Due to the above-mentioned features, the present invention is able to provide simultaneous improvements in terms of lack of stickiness, smooth texture, and fitting sensation when applied, which were considered to be problems in water-in-oil emulsion cosmetics. Additionally, aside therefrom, the feeling in use, such as the wateriness and the spreadability when applied, can be improved.

In the present invention, a “fitting sensation” refers to a sensation in which, when the cosmetic is applied, there are no situations in which the cosmetic keeps spreading without finishing, and there is a good sense of the cosmetic being taken up by the skin.

### MODES FOR CARRYING OUT THE INVENTION

The present invention is a water-in-oil emulsion cosmetic that contains (A) a water-soluble thickener in an internal phase, and contains at least one of (B) an emulsifying crosslinked elastomer and (C) a metal soap in an external phase. The respective components will be explained in detail below.

### <(A) Water-Soluble Thickener>

The water-soluble thickener is a water-soluble thickener that can normally be used in cosmetics.

Water-soluble thickeners include, for example, plant-based polymers such as gum arabic, tragacanth, galactan, carob gum, guar gum, karaya gum, carrageenan, xanthan gum, pectin, agar, quince seed (marmelo) and algecolloid (Phaeophyceae extract); microbe-based polymers such as gellan gum, dextran, succinoglucan and pullulan; animal-based polymers such as collagen, casein, albumin and gelatin; and starch-based polymers such as starch (rice, corn, potato, wheat), carboxymethyl starch and methyl hydroxypropyl starch.

Additional examples include cellulose-based polymers such as methyl cellulose, nitrocellulose, ethyl cellulose, methyl hydroxypropyl cellulose, hydroxyethyl cellulose, sodium cellulose sulfate, hydroxypropyl cellulose, sodium carboxymethyl cellulose, crystalline cellulose and cellulose powder; and alginic acid-based polymers such as sodium alginate and propylene glycol esters of alginic acid.

Further examples include vinyl-based polymers such as polyvinyl methyl ether and carboxyvinyl polymers; polyoxyethylene-based polymers; polyoxyethylene/polyoxypropylene copolymers; acryl-based polymers such as poly ethyl acrylate and polyacrylamide; polyethyleneimine; cationic polymers; inorganic water-soluble polymers such as bentonite, aluminum-magnesium silicate, laponite, hectorite and silicic anhydride; PEG-240/decyltetradeceth-20/hexamethylene diisocyanate copolymer; (dimethylacrylamide/sodium acryloyldimethyl taurate) crosspolymer, (sodium acrylate/acryloyldimethyl taurate) copolymer, (alkyl acrylate/steareth-20 methacrylate) copolymer and (ammonium acryloyldimethyl taurate/VP) copolymer.

Among the above, a polysaccharide-based thickener should preferably be blended as the water-soluble thickener due to having excellent smoothness, and it is particularly preferable to blend a combination of two or more types of polysaccharide-based thickeners.

Examples of polysaccharide-based thickeners include cellulose-based thickeners such as crystalline cellulose, methyl cellulose, ethyl cellulose, hydroxyethyl cellulose, hydroxypropyl cellulose and carboxymethyl cellulose; and natural polymeric polysaccharide thickeners such as gellan gum, carrageenan, guar gum, locust bean gum, gum arabic, xanthan gum, dextran and succinoglucan.

A particularly preferred polysaccharide-based thickener is a crystalline cellulose known as cellulose nanofibers, which are obtained by fibrillating cellulose fibers to the nanometer level. The cellulose nanofibers preferably have a fiber width of 10 nm or less, more preferably 5 nm or less.

The cellulose nanofibers can be obtained by mechanical fibrillation. Preferably, prior to the fibrillation, the cellulose is catalytically oxidized with TEMPO (2,2,6,6-tetramethylpiperidine-1-oxy radicals), thereby introducing carboxy groups (sodium carboxylate) to the cellulose microfibril surfaces. Due to this pretreatment, the electric double-layer repulsive force can be utilized to obtain microfibril units with lighter mechanical fibrillation.

The cellulose fibers may be of a type that is commercially available as an aqueous dispersion having water or a water-miscible organic solvent as the dispersion medium. An example of such a commercial product is “Rheocrysta C-2SP (DKS Co., Ltd.)” or the like.

The water-soluble thickener is dispersed in the internal phase of the water-in-oil emulsion cosmetic to thicken the internal phase. The blended amount of the water-soluble thickener is 0.01% to 15% by mass, preferably 0.1% to 5% by mass, and more preferably 0.5% to 2% by mass relative to the total amount of the water-in-oil emulsion cosmetic. If the blended amount of the water-soluble thickener is too small, then there are cases in which the improvement in smoothness and the like cannot be adequately sensed. Conversely, if the blended amount is too high, then it becomes difficult to disperse the water-soluble thickener in the water phase.

### <(B) Emulsifying Crosslinked Elastomer and (C) Metal Soap>

The emulsifying crosslinked elastomer and the metal soap emulsify, thicken or gel the oil phase, thereby increasing the stability of the water-in-oil emulsion cosmetic. Of the emulsifying crosslinked elastomer and the metal soap, one may be blended alone, or both may be blended in combination.

### (B) Emulsifying Crosslinked Elastomer

The emulsifying crosslinked elastomer is an elastomer provided with hydrophilic chains and a crosslinked structure.

The emulsifying crosslinked elastomer is, for example, a crosslinked silicone that is crosslinked by polyether chains or polyglycerin chains, and may be optionally selected from among those having branched chains such as alkyl chains or silicone chains.

Examples of emulsifying crosslinked elastomers include:
(dimethicone/(PEG-10/15)) crosspolymers, which are crosslinked silicones that are crosslinked by polyether chains;
(PEG-15/lauryl dimethicone) crosspolymers, which are polyether-modified crosslinked silicones having alkyl chains in the structures;
(PEG-15/lauryl polydimethylsiloxyethyl dimethicone) crosspolymers, which are polyether-modified crosslinked silicones having two types of branched chains, namely, silicone chains and alkyl chains;
(dimethicone/polyglycerin-3) crosspolymers, which are crosslinked silicones that are crosslinked by polyglycerin chains;
(lauryl dimethicone/polygylcerin-3) crosspolymers, which are polyglycerin-modified crosslinked silicones having alkyl chains in the structures; and
(polglyceryl-3/lauryl polydimethylsiloxyethyl dimethicone) crosspolymers, which are polyglycerin-modified crosslinked silicones having two types of branched chains, namely, silicone chains and alkyl chains.

Commercially available products that are emulsifying crosslinked elastomers include, for example, KSG-210, KSG-310, KSG-360Z, KSG-710 (all of the above from Shin-Etsu Chemical Co., Ltd.) and the like.

### (C) Metal Soap

The metal soap is a metal salt of a saturated or unsaturated higher fatty acid, and is not particularly limited. However, it should preferably be an aluminum, calcium, magnesium or zinc salt of a saturated and/or unsaturated fatty acid having 8 to 24, preferably 12 to 18, carbon atoms.

Metal soaps include, for example, aluminum distearate, aluminum myristate, magnesium myristate, zinc stearate, zinc myristate and zinc oleate.

In the case in which an emulsifying crosslinked elastomer is blended into the water-in-oil emulsion cosmetic of the present invention, the amount thereof should be 0.01% to 30% by mass, preferably 1% to 10% by mass relative to the total amount of the water-in-oil emulsion cosmetic. If the blended amount of the emulsifying crosslinked elastomer is too small, then there are cases in which the stability becomes lower. Conversely, if the blended amount is too high, then there is a tendency for the stickiness to increase significantly.

Meanwhile, in the case in which a metal soap is blended, the amount thereof should be 0.01% to 10% by mass, preferably 1% to 5% by mass relative to the total amount of the water-in-oil emulsion cosmetic. If the blended amount of the metal soap is too small, then there are cases in which the stability becomes lower. Conversely, if the blended amount is too high, then the cosmetic becomes heavy to spread at the time of use and there is a tendency for the stickiness to increase significantly.

Additionally, in the case in which a combination of an emulsifying crosslinked elastomer and a metal soap is blended, the blended amount mentioned above should be appropriately adjusted in accordance with the blended amount of the other.

### <Optional Blended Components>

Aside from the above-mentioned components (A) to (C), components that are normally used in cosmetics may be blended into the water-in-oil emulsion cosmetic of the present invention within a range not hindering the effects of the present invention. For example, various types of solvents, surfactants, powder components, medicinal agents, ultraviolet absorption agents, antioxidants, metal sequestrants, pH adjusters, fragrances, preservatives and the like may be appropriately blended as needed.

In particular, the water-in-oil emulsion cosmetic according to the present invention has excellent stability while also suppressing stickiness and the like. Thus, powder components can be stably dispersed therein. The powder components that may be blended are not particularly limited. Examples include organic resin powders such as polyamide resin powders (nylon powders, etc.), polyethylene powders, polymethyl methacrylate powders, polystyrene powders, styrene and acrylic acid copolymer resin powders and cellulose powders; silicone resin powders such as polymethyl silsesquioxane powders; silicone rubber powders such as polydimethyl siloxane crosslinked elastomers; hydrophobic silica powders such as dimethyl silylated silicic anhydride and trimethyl silylated silicic anhydride; dimethyl siloxane-treated powders such as dimethyl polysiloxane-treated titanium dioxide, similarly treated mica and similarly treated talc and similarly treated iron oxide; and hydrophobically surface-treated powders such as dextrin palmitate-treated powders including dextrin palmitate-treated talc, similarly treated mica, similarly treated titanium dioxide and similarly treated iron oxide.

When blending a powder component, the blended amount thereof should preferably be 0.1% to 30% by mass, more preferably 5% to 20% by mass relative to the total amount of the water-in-oil emulsion cosmetic. If the blended amount of the powder component is too high, then there is a tendency for the spreadability when applied and the like to become worse.

### <Preparation and Usage>

The water-in-oil emulsion cosmetic of the present invention can be prepared by means of conventional methods, and the emulsification method is not particularly limited. An example of such a method is one that involves heating each of a water phase containing (A) a water-soluble thickener, and an oil phase containing at least one of (B) an emulsifying crosslinked elastomer and (C) a metal soap to approximately 70° C., thereafter adding the water phase to the oil phase little by little, emulsifying the mixture with an emulsifying machine, and allowing the emulsion to cool to room temperature. However, the method is not limited thereto.

The water-in-oil emulsion cosmetic according to the present invention can be widely applied to cosmetics and, for example, may be an emulsified foundation, a sun-care cosmetic, a makeup base or the like.

Additionally, for the present invention, the form of the container is not limited. For example, an impregnated body may be impregnated with this cosmetic, then housed inside a compact container that is airtight. Examples of the impregnated body include nonwoven fabrics comprising single or mixed materials such as resin, pulp and cotton, resin-treated fibrous bodies, foamed bodies such as sponges, and porous bodies provided with continuous pores. Additionally, examples of the material of the impregnated body include NBR (acrylonitrile butadiene rubber), SBR (styrene butadiene rubber), NR (natural rubber), urethane, nylon, polyolefin, polyester, EVA (ethylene vinyl acetate), PVA (polyvinyl alcohol), silicone, elastomers and the like. However, the impregnated body is not limited to being composed of these materials as long as the impregnated body is able to contain the cosmetic.

Examples of the applicator for taking the cosmetic from the impregnated body and applying the cosmetic to skin include sponges, puffs, tips and the like that are normally used when applying liquid cosmetics to skin.

Additionally, the water-in-oil emulsion cosmetic according to the present invention can be contained as a base liquid in an aerosol cosmetic. The propellant is not particularly limited as long as it is a propellant that is normally used in aerosol cosmetics. Examples include liquefied petroleum gas, dimethyl ether, nitrogen, oxygen, carbon dioxide, chlorofluorocarbons and the like. The aerosol cosmetic can be produced by filling an aerosol can with one or more of these propellants, together with the base liquid, by means of a conventional method.

### EXAMPLES

Although the present invention will be explained in further detail by providing examples below, the present invention is not limited in any way thereby. Where not otherwise noted, the blended amounts represent the percentages by mass of the component relative to the total amount of the cosmetic. Before specifically explaining each example, the evaluation methods that were employed will be explained.

The water-in-oil emulsion cosmetics having the compositions listed in Table 1 below were prepared by heating and dissolving the oil-based components, dispersing the powders therein, adding the separately dissolved water phase thereto, then emulsifying the mixture by stirring. The cosmetics that were obtained were subjected to actual usage tests by ten expert panelists, and evaluated for smoothness, lack of stickiness and fitting sensation in accordance with the evaluation criteria below.

### <Evaluation Criteria>

A: Nine or more panelists evaluated the cosmetic as being good
B: Six to eight panelists evaluated the cosmetic as being good
C: Three to five panelists evaluated the cosmetic as being good
D: Two or fewer panelists evaluated the cosmetic as being good

TABLE 1

| | | | | | | | | | | | Co | Co | Co | Co |
|---|---|---|---|---|---|---|---|---|---|---|---|---|---|---|
| | | Ex 1 | Ex 2 | Ex 3 | Ex 4 | Ex 5 | Ex 6 | Ex 7 | Ex 8 | Ex 9 | Ex 1 | Ex 2 | Ex 3 | Ex 4 |
| Dimethicone | | 20 | 20 | 20 | 20 | 20 | 20 | 20 | 20 | 20 | 20 | 20 | 20 | 20 |
| Diphenylsiloxyphenyl | | 10 | 10 | 10 | 10 | 10 | 10 | 10 | 10 | 10 | 10 | 10 | 10 | 10 |
| trimethicone | | | | | | | | | | | | | | |
| PEG-10 dimethicone | | 4 | 4 | 4 | 4 | 4 | 4 | 4 | 4 | 4 | 4 | 4 | 4 | 4 |
| Sorbitan sesquiisostearate | | 2 | 2 | 2 | 2 | 2 | 2 | 2 | 2 | 2 | 2 | 2 | 2 | 2 |
| (PEG-15/lauryl | | — | 5 | — | — | — | — | — | — | — | — | — | — | — |
| polydimethylsiloxyethyl | | | | | | | | | | | | | | |
| dimethicone) crosspolymer ^{*1} | | | | | | | | | | | | | | |
| Zinc myristate | | 0.5 | 0.5 | — | — | 0.5 | 0.5 | 0.5 | 0.5 | 0.5 | — | — | — | 0.5 |
| Aluminum distearate | | — | — | 0.5 | — | — | — | — | — | — | — | — | — | — |
| Magnesium myristate | | — | — | — | 0.5 | — | — | — | — | — | — | — | — | — |
| Dextrin palmitate | | — | — | — | — | — | — | — | — | — | — | 0.5 | — | — |
| Disteardimonium hectorite | | — | — | — | — | — | — | — | — | — | — | — | 0.5 | — |
| Hydrophobically | | 10 | 10 | 10 | 10 | 10 | 10 | 10 | 10 | 10 | 10 | 10 | 10 | 10 |
| treated titanium oxide | | | | | | | | | | | | | | |
| Hydrophobically | | 0.7 | 0.7 | 0.7 | 0.7 | 0.7 | 0.7 | 0.7 | 0.7 | 0.7 | 0.7 | 0.7 | 0.7 | 0.7 |
| treated red iron oxide | | | | | | | | | | | | | | |
| Hydrophobically | | 1.5 | 1.5 | 1.5 | 1.5 | 1.5 | 1.5 | 1.5 | 1.5 | 1.5 | 1.5 | 1.5 | 1.5 | 1.5 |
| treated yellow iron oxide | | | | | | | | | | | | | | |
| Hydrophobically | | 0.1 | 0.1 | 0.1 | 0.1 | 0.1 | 0.1 | 0.1 | 0.1 | 0.1 | 0.1 | 0.1 | 0.1 | 0.1 |
| treated black iron oxide | | | | | | | | | | | | | | |
| Water | | 45 | 40 | 45 | 45 | 45.98 | 45 | 45.8 | 45.9 | 44.98 | 45.5 | 45 | 45 | 46 |
| Glycerin | | 5 | 5 | 5 | 5 | 5 | 5 | 5 | 5 | 5 | 5 | 5 | 5 | 5 |
| Preservative | | 0.2 | 0.2 | 0.2 | 0.2 | 0.2 | 0.2 | 0.2 | 0.2 | 0.2 | 0.2 | 0.2 | 0.2 | 0.2 |
| Cellulose nanofibers | | 1 | 1 | 1 | 1 | — | — | — | — | 1 | 1 | 1 | 1 | — |
| Hydroxyethyl cellulose | | — | — | — | — | 0.02 | — | — | — | 0.02 | — | — | — | — |
| Agar | | — | — | — | — | — | 1 | — | — | — | — | — | — | — |
| Gellan gum | | — | — | — | — | — | — | 0.2 | — | — | — | — | — | — |
| (Dimethyl acrylamide/sodium | | — | — | — | — | — | — | — | 0.1 | — | — | — | — | — |
| acryloyldimethyl taurate) | | | | | | | | | | | | | | |
| crosspolymer | | | | | | | | | | | | | | |
| Total | | 100 | 100 | 100 | 100 | 100 | 100 | 100 | 100 | 100 | 100 | 100 | 100 | 100 |
| Evaluation | Fitting sensation | B | A | B | B | A | A | A | A | A | B | B | B | C |
| | Smoothness | A | A | A | A | A | A | A | A | A | C | C | B | B |
| | Lackofstickiness | A | A | A | A | A | A | A | B | A | A | D | C | A |

^{*1} KSG-360Z (Shin-etsu Chemical Co., Ltd.) solid part 30% to 40%

As indicated in Table 1, in the case in which a water-soluble thickener was blended into the water phase, and at least one of an emulsifying crosslinked elastomer and a metal soap was blended into the oil phase, extremely excellent results were obtained in all evaluation categories (Examples 1 to 9). In particular, in the case in which zinc myristate was blended as a metal soap, a tendency to have an excellent fitting sensation was observed, irrespective of the type of water-soluble thickener that was combined. Additionally, a tendency to have excellent properties in terms of lack of stickiness was observed more in the case in which a polysaccharide-based thickener was blended as the water-soluble thickener than in the case in which a synthetic polymer thickener was blended.

Conversely, the smoothness was poorer in the case in which neither a metal soap nor an emulsifying crosslinked elastomer was contained in the oil phase (Comparative Example 1), and in the case in which a water-soluble thickener was not contained, the lack of stickiness was excellent but the fitting sensation was poor (Comparative Example 4). Additionally, in the case in which an oil-soluble thickener that is generally used in cosmetics was blended instead of a metal soap, extreme stickiness occurred (Comparative Examples 2 and 3).

The water-in-oil emulsion cosmetics having the compositions listed in Table 2 below were prepared by heating and dissolving the oil-based components, dispersing the powders therein, adding the separately dissolved water phase thereto, then emulsifying the mixture by stirring. The cosmetics that were obtained were subjected to actual usage tests by ten expert panelists, and evaluated for smoothness, lack of stickiness, fitting sensation, wateriness and spreadability when applied, in accordance with the evaluation criteria below.

### <Evaluation Criteria>

A: Nine or more panelists evaluated the cosmetic as being good
B: Six to eight panelists evaluated the cosmetic as being good
C: Three to five panelists evaluated the cosmetic as being good
D: Two or fewer panelists evaluated the cosmetic as being good

TABLE 2

| | | | | | | | | | Co | Co | Co |
|---|---|---|---|---|---|---|---|---|---|---|---|
| | | Ex 10 | Ex 11 | Ex 12 | Ex 13 | Ex 14 | Ex 15 | Ex 16 | Ex 5 | Ex 6 | Ex 7 |
| Dimethicone | | 15 | 20 | 20 | 20 | 20 | 20 | 20 | 20 | 20 | 20 |
| Diphenylsiloxyphenyl | | 10 | 10 | 10 | 10 | 10 | 10 | 10 | 10 | 10 | 10 |
| trimethicone | | | | | | | | | | | |
| PEG-10 dimethicone | | 4 | 4 | 4 | 4 | 4 | 4 | 4 | 4 | 4 | 4 |
| (PEG-15/lauryl | | 5 | — | 5 | 5 | 5 | 5 | 5 | 5 | — | — |
| polydimethylsiloxyethyl | | | | | | | | | | | |
| dimethicone) crosspolymer ^{*1} | | | | | | | | | | | |
| Sorbitan sesquiisostearate | | 2 | 2 | 2 | 2 | 2 | 2 | 2 | 2 | 2 | 2 |
| Zinc myristate | | — | 0.5 | 0.5 | 0.5 | 0.5 | 0.5 | — | 0.5 | 0.5 | — |
| Aluminum distearate | | — | — | — | — | — | — | 0.5 | — | — | — |
| Disteardimonium hectorite | | — | — | — | — | — | — | — | — | 0.5 | — |
| Dextrin palmitate | | — | — | — | — | — | — | — | — | — | 0.5 |
| Hydrophobically | | 10 | 10 | 10 | 10 | 10 | 10 | 10 | 10 | 10 | 10 |
| treated titanium oxide | | | | | | | | | | | |
| Hydrophobically | | 0.7 | 0.7 | 0.7 | 0.7 | 0.7 | 0.7 | 0.7 | 0.7 | 0.7 | 0.7 |
| treated red iron oxide | | | | | | | | | | | |
| Hydrophobically | | 1.5 | 1.5 | 1.5 | 1.5 | 1.5 | 1.5 | 1.5 | 1.5 | 1.5 | 1.5 |
| treated yellow iron oxide | | | | | | | | | | | |
| Hydrophobically | | 0.1 | 0.1 | 0.1 | 0.1 | 0.1 | 0.1 | 0.1 | 0.1 | 0.1 | 0.1 |
| treated black iron oxide | | | | | | | | | | | |
| Water | | 46.4 | 45.9 | 40.9 | 40.8 | 40.8 | 40.8 | 40.8 | 41 | 45.5 | 45.8 |
| Glycerin | | 5 | 5 | 5 | 5 | 5 | 5 | 5 | 5 | 5 | 5 |
| Preservative | | 0.2 | 0.2 | 0.2 | 0.2 | 0.2 | 0.2 | 0.2 | 0.2 | 0.2 | 0.2 |
| Cellulose nanofibers | | 0.1 | 0.1 | 0.1 | 0.1 | 0.1 | 0.1 | 0.1 | — | — | — |
| Hydroxyethyl cellulose | | — | — | — | 0.1 | — | — | 0.1 | — | — | 0.2 |
| Gellan gum | | — | — | — | — | 0.1 | — | — | — | — | — |
| (Dimethyl acrylamide/sodium | | — | — | — | — | — | 0.1 | — | — | — | — |
| acryloyldimethyl taurate) | | | | | | | | | | | |
| crosspolymer | | | | | | | | | | | |
| Total | | 100 | 100 | 100 | 100 | 100 | 100 | 100 | 100 | 100 | 100 |
| Evaluation | Wateriness | A | A | A | A | A | B | A | D | D | D |
| | Smoothness | A | A | A | A | A | A | A | D | B | C |
| | Lack of stickiness | A | A | A | A | A | B | A | B | B | D |
| | Spreadability | A | A | A | A | A | A | A | D | B | C |
| | when applied | | | | | | | | | | |
| | Fitting sensation | A | A | A | A | A | A | A | D | B | B |

^{*1} KSG-360Z (Shin-etsu Chemical Co., Ltd.) solid part 30% to 40%

As indicated in Table 2, when cellulose nanofibers were blended, as a water-soluble thickener, into the water phase and at least one of an emulsifying crosslinked elastomer and a metal soap was blended into the oil phase, extremely good results were able to be obtained in all evaluation categories (Examples 10 to 16).

Conversely, satisfactory results were not able to be obtained in multiple evaluation categories in the case in which a water-soluble thickener was not contained, and in the cases in which other oil-soluble thickeners that are generally used in cosmetics were blended instead of an emulsifying crosslinked elastomer and a metal soap (Comparative Examples 5 to 7).

Hereinafter, examples of formulations of the cosmetic of the present invention will be indicated. Needless to say, the present invention is not limited in any way by these formulation examples, and is as defined by the claims. The blended amounts are all indicated in percentage by mass relative to the total amount of the cosmetic.

### Formulation Example 1: Foundation

| (Component | Blended amount |
|---|---|
| name) | (% by mass) |
| Dimethicone | 20 |
| Diphenylsiloxyphenyl trimethicone | 10 |
| PEG-10 dimethicone | 4 |
| (PEG-15/lauryl | 5 |
| polydimethylsiloxyethyl dimethicone) | |
| crosspolymer (KSG-360Z (Shin-etsu | |
| Chemical Co., Ltd.) solid part 30% to 40%) | |
| Sorbitan sesquiisostearate | 2 |
| Ethylhexyl methoxycinnamate | 5 |
| Zinc myristate | 0.5 |
| (Vinyl dimethicone/methicone | 2 |
| silsesquioxane) crosspolymer | |
| Nylon-12 | 2 |
| Dimethyl silylated silica | 1 |
| Hydrophobically treated | 10 |
| pigment-grade titanium | |
| Hydrophobically treated | 0.7 |
| red iron oxide | |
| Hydrophobically treated | 1.5 |
| yellow iron oxide | |
| Hydrophobically treated | 0.1 |
| black iron oxide | |
| Water | 29.98 |
| Glycerin | 5 |
| Preservative | 0.2 |
| Cellulose nanofibers | 1 |
| Hydroxyethyl cellulose | 0.02 |

### Formulation Example 2: Concealer

| (Component name) | Blended amount (% by mass) |
|---|---|
| Dimethicone | 20 |
| Diphenylsiloxyphenyl trimethicone | 10 |
| PEG-10 dimethicone | 4 |
| (Dimethicone/(PEG-10/ | 5 |
| 15)) crosspolymer | |
| Sorbitan sesquiisostearate | 2 |
| Zinc myristate | 0.5 |
| (Vinyl dimethicone/methicone | 3 |
| silsesquioxane) crosspolymer | |
| Nylon-12 | 2 |
| Hydrophobically treated | 12 |
| pigment-grade titanium | |
| Hydrophobically treated | 0.7 |
| red iron oxide | |
| Hydrophobically treated | 1.5 |
| yellow iron oxide | |
| Hydrophobically treated | 0.1 |
| black iron oxide | |
| Water | 32.98 |
| Glycerin | 5 |
| Preservative | 0.2 |
| Cellulose nanofibers | 1 |
| Hydroxyethyl cellulose | 0.02 |

### Formulation Example 3: BB Cream

| (Component | Blended amount |
|---|---|
| name) | (% by mass) |
| Dimethicone | 20 |
| Diphenylsiloxyphenyl trimethicone | 10 |
| PEG-10 dimethicone | 2 |
| (Dimethicone/polyglycerin-3) | 5 |
| crosspolymer | |
| Lauryl PEG-9 | 2 |
| polydimethylsiloxyethyl | |
| dimethicone | |
| Sorbitan sesquiisostearate | 2 |
| Ethylhexyl methoxycinnamate | 3 |
| Zinc myristate | 0.5 |
| (Vinyl dimethicone/methicone | 2 |
| silsesquioxane) crosspolymer | |
| Nylon-12 | 2 |
| Dimethyl silylated silica | 0.5 |
| Hydrophobically treated | 6 |
| pigment-grade titanium | |
| Hydrophobically treated | 0.7 |
| red iron oxide | |
| Hydrophobically treated | 1.5 |
| yellow iron oxide | |
| Hydrophobically treated | 0.1 |
| black iron oxide | |
| Water | 36.48 |
| Glycerin | 5 |
| Preservative | 0.2 |
| Cellulose nanofibers | 1 |
| Hydroxyethyl cellulose | 0.02 |

### Formulation Example 4: Eyeshadow

| (Component | Blended amount |
|---|---|
| name) | (% by mass) |
| Dimethicone | 20 |
| Diphenylsiloxyphenyl trimethicone | 10 |
| PEG-10 dimethicone | 4 |
| (PEG-15/lauryl | 5 |
| polydimethylsiloxyethyl | |
| dimethicone) crosspolymer | |
| (KSG-360Z (Shin-etsu | |
| Chemical Co., Ltd.) solid | |
| part 30% to 40%) | |
| Sorbitan sesquiisostearate | 3 |
| Zinc myristate | 0.5 |
| Silicone-treated red iron | 5 |
| oxide-coated titanated mica | |
| Silicone-treated red iron | 5 |
| oxide-coated mica | |
| Hydrophobically treated | 10 |
| pigment-grade titanium | |
| Hydrophobically treated | 0.7 |
| red iron oxide | |
| Hydrophobically treated | 1.5 |
| yellow iron oxide | |
| Hydrophobically treated | 0.1 |
| black iron oxide | |
| Water | 28.98 |
| Glycerin | 5 |
| Preservative | 0.2 |
| Cellulose nanofibers | 1 |
| Hydroxyethyl cellulose | 0.02 |

### Formulation Example 5: Skin-Care Cream

| (Component | Blended amount |
|---|---|
| name) | (% by mass) |
| Dimethicone | 20 |
| Diphenylsiloxyphenyl trimethicone | 10 |
| PEG-10 dimethicone | 4 |
| (PEG-15/lauryl | 5 |
| polydimethylsiloxyethyl | |
| dimethicone) crosspolymer | |
| (KSG-360Z (Shin-etsu | |
| Chemical Co., Ltd.) solid part 30% to 40%) | |
| Zinc myristate | 0.5 |
| (Vinyl dimethicone/methicone | 2 |
| silsesquioxane) crosspolymer | |
| Water | 51.28 |
| Glycerin | 5 |
| Preservative | 0.2 |
| Cellulose nanofibers | 1 |
| Hydroxyethyl cellulose | 0.02 |

### Formulation Example 6: Sunscreen

| (Component | Blended amount |
|---|---|
| name) | (% by mass) |
| Dimethicone | 20 |
| Diphenylsiloxyphenyl trimethicone | 10 |
| PEG-10 dimethicone | 4 |
| (PEG-15/lauryl | 5 |
| polydimethylsiloxyethyl dimethicone) | |
| crosspolymer (KSG-360Z (Shin-etsu | |
| Chemical Co., Ltd.) solid part 30% to 40%) | |
| Sorbitan sesquiisostearate | 2 |
| Ethylhexyl methoxycinnamate | 7.5 |
| Zinc myristate | 0.5 |
| Hydrophobically treated | 3 |
| zinc oxide | |
| Hydrophobically treated | 8 |
| pigment-grade titanium | |
| Hydrophobically treated | 5 |
| fine-particle titanium | |
| Hydrophobically treated | 0.7 |
| red iron oxide | |
| Hydrophobically treated | 1.5 |
| yellow iron oxide | |
| Hydrophobically treated | 0.1 |
| black iron oxide | |
| Water | 26.48 |
| Glycerin | 5 |
| Preservative | 0.2 |
| Cellulose nanofibers | 1 |
| Hydroxyethyl cellulose | 0.02 |

1. A water-in-oil emulsion cosmetic that:
contains (A) a water-soluble thickener in an internal phase; and
contains at least one of (B) an emulsifying crosslinked elastomer and (C) a metal soap in an external phase.

2. The water-in-oil emulsion cosmetic as in claim 1, wherein the (A) water-soluble thickener is a polysaccharide thickener.

3. The water-in-oil emulsion cosmetic as in claim 1, containing two or more types of polysaccharide thickeners as the (A) water-soluble thickener.

4. The water-in-oil emulsion cosmetic as in claim 1, containing cellulose nanofibers as the (A) water-soluble thickener.

5. The water-in-oil emulsion cosmetic as in claim 1, wherein the (B) emulsifying crosslinked elastomer is a crosslinked silicone that is crosslinked by polyether chains or polyglycerin chains.

6. The water-in-oil emulsion cosmetic as in claim 1, wherein the (C) metal soap is zinc myristate.
